SEQUENCE LISTING (11 sequences)

SEQ ID NO: 1 - protein, 133 aa, Homo sapiens [sequence omitted]

SEQ ID NO: 2 - DNA, 399 bp, Homo sapiens
  source 1..399: mol_type = genomic DNA
caggggcaagaccgacacatgattaggatgcgccaactgatagacatagtcgaccaattgaaaaactatgtaaacgacttggtgcctgagttcctgcctgcgccggaagatgtcgaaaccaattgcgagtggtccgccttctcatgctttcagaaggcgcagttgaaaagtgccaacacagggaataacgaaaggataattaacgtgagtataaagaagttgaaacggaaacctccaagcacgaacgcgggtcgacgccagaagcaccgattgacgtgcccttcttgcgactcctacgaaaaaaagccacctaaagagttcctggagaggttcaagtctctgctccagaaaatgatacatcagcatctgagcagcagaacgcatggctccgaggattcc

SEQ ID NO: 3 - protein, 538 aa, Homo sapiens
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 4 - DNA, 1614 bp, Homo sapiens
  source 1..1614: mol_type = genomic DNA
atgccgaggggttgggcagcccctcttctcctgctgctgctccaaggaggttggggctgtcccgatctcgtctgctataccgattatctgcagacggttatatgtatactcgaaatgtggaatctccacccatctacgcttactctcacatggcaagaccagtacgaggagcttaaagacgaagccacgagttgctccctccaccggtcagcgcacaacgccacccatgcgacgtacacttgtcatatggacgtatttcactttatggctgatgatattttctcagttaacatcacagaccagtctggtaattatagccaggagtgtgggtcattccttctggcggagtctataaaaccagcgccgcccttcaacgtcactgtaacattctcagggcaatataacatctcttggcggagtgactacgaagatccagccttttacatgttgaagggaaagttgcagtatgagttgcagtacaggaaccgcggagatccctgggcggtatcacctcggaggaagctgataagcgtcgatagccgctctgtgagtctcctgcctcttgaattccgaaaagattcctcttacgagctccaagttagggcgggacccatgccaggcagctcttaccaaggtacctggtctgaatggagtgacccagttatcttccagactcaatcagaagaactgaaggaagggtggaatccgcatctgttgttgctgctcctcctcgtaatagttttcatacctgccttttggagcctgaagacgcaccctttgtggagactctggaagaaaatctgggcagttccgagtcccgagcgcttctttatgccactttataagggctgcagtggtgattttaaaaaatgggtaggtgccccgtttaccggttctagcttggagcttggaccttggtccccagaggtaccttccactttggaagtctacagctgccaccccccgcggagtccagccaagcgattgcaactgacggagttgcaggaaccagcagaacttgtcgaaagcgacggcgttcctaagccatctttttggcctaccgcgcaaaatagtgggggaagtgcttactcagaagagagagaccggccctatggactcgtttcaattgatacagttacggttcttgacgccgaaggaccatgtacttggccatgtagttgcgaagacgatggatatccagcactcgatctcgatgcagggttggaaccttccccaggccttgaagatccccttcttgacgcgggaacaacagttctgtcctgtgggtgtgtttccgcgggttcccctggacttggaggcccacttggatcccttctggacagattgaagcccccactcgccgatggagaggactgggcaggcgggctgccgtggggcgggaggtcacctgggggcgtttcagaaagtgaagcggggtctccactcgctgggttggatatggatacctttgacagcggttttgtgggtagtgactgttcatcccccgtcgagtgcgatttcacttctccgggagacgaaggacccccgcggtcttaccttagacagtgggttgtaattcctccaccgcttagctctcctggtccccaggcgagt

SEQ ID NO: 5 - protein, 26 aa, synthetic construct
SGGGSGGGGSGGGGSGGGGSGGGSLQ

SEQ ID NO: 6 - DNA, 78 bp, synthetic construct
  source 1..78: mol_type = other DNA
tcaggcggtgggtcaggcggaggtgggtctggaggtggaggtagtggaggtggaggatcaggcggggggtcccttcaa

SEQ ID NO: 7 - protein, 24 aa, Homo sapiens
MSFPCKFVASFLLIFNVSSKGAVS

SEQ ID NO: 8 - DNA, 72 bp, Homo sapiens
  source 1..72: mol_type = genomic DNA
atgtcattcccttgtaaattcgtggcatccttcctcctgatattcaatgtaagttcaaagggggccgtgtcc

SEQ ID NO: 9 - protein, 721 aa, synthetic construct
MSFPCKFVASFLLIFNVSSKGAVSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSSGGGSGGGGSGGGGSGGGGSGGGSLQMPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 10 - DNA, 2163 bp, synthetic construct
  source 1..2163: mol_type = other DNA
atgtcattcccttgtaaattcgtggcatccttcctcctgatattcaatgtaagttcaaagggggccgtgtcccaggggcaagaccgacacatgattaggatgcgccaactgatagacatagtcgaccaattgaaaaactatgtaaacgacttggtgcctgagttcctgcctgcgccggaagatgtcgaaaccaattgcgagtggtccgccttctcatgctttcagaaggcgcagttgaaaagtgccaacacagggaataacgaaaggataattaacgtgagtataaagaagttgaaacggaaacctccaagcacgaacgcgggtcgacgccagaagcaccgattgacgtgcccttcttgcgactcctacgaaaaaaagccacctaaagagttcctggagaggttcaagtctctgctccagaaaatgatacatcagcatctgagcagcagaacgcatggctccgaggattcctcaggcggtgggtcaggcggaggtgggtctggaggtggaggtagtggaggtggaggatcaggcggggggtcccttcaaatgccgaggggttgggcagcccctcttctcctgctgctgctccaaggaggttggggctgtcccgatctcgtctgctataccgattatctgcagacggttatatgtatactcgaaatgtggaatctccacccatctacgcttactctcacatggcaagaccagtacgaggagcttaaagacgaagccacgagttgctccctccaccggtcagcgcacaacgccacccatgcgacgtacacttgtcatatggacgtatttcactttatggctgatgatattttctcagttaacatcacagaccagtctggtaattatagccaggagtgtgggtcattccttctggcggagtctataaaaccagcgccgcccttcaacgtcactgtaacattctcagggcaatataacatctcttggcggagtgactacgaagatccagccttttacatgttgaagggaaagttgcagtatgagttgcagtacaggaaccgcggagatccctgggcggtatcacctcggaggaagctgataagcgtcgatagccgctctgtgagtctcctgcctcttgaattccgaaaagattcctcttacgagctccaagttagggcgggacccatgccaggcagctcttaccaaggtacctggtctgaatggagtgacccagttatcttccagactcaatcagaagaactgaaggaagggtggaatccgcatctgttgttgctgctcctcctcgtaatagttttcatacctgccttttggagcctgaagacgcaccctttgtggagactctggaagaaaatctgggcagttccgagtcccgagcgcttctttatgccactttataagggctgcagtggtgattttaaaaaatgggtaggtgccccgtttaccggttctagcttggagcttggaccttggtccccagaggtaccttccactttggaagtctacagctgccaccccccgcggagtccagccaagcgattgcaactgacggagttgcaggaaccagcagaacttgtcgaaagcgacggcgttcctaagccatctttttggcctaccgcgcaaaatagtgggggaagtgcttactcagaagagagagaccggccctatggactcgtttcaattgatacagttacggttcttgacgccgaaggaccatgtacttggccatgtagttgcgaagacgatggatatccagcactcgatctcgatgcagggttggaaccttccccaggccttgaagatccccttcttgacgcgggaacaacagttctgtcctgtgggtgtgtttccgcgggttcccctggacttggaggcccacttggatcccttctggacagattgaagcccccactcgccgatggagaggactgggcaggcgggctgccgtggggcgggaggtcacctgggggcgtttcagaaagtgaagcggggtctccactcgctgggttggatatggatacctttgacagcggttttgtgggtagtgactgttcatcccccgtcgagtgcgatttcacttctccgggagacgaaggacccccgcggtcttaccttagacagtgggttgtaattcctccaccgcttagctctcctggtccccaggcgagt

SEQ ID NO: 11 - protein, 5 aa, unidentified
  VARIANT 3: note = Any amino acid
WSXWS

(19) United States
(12) Patent Application Publication
(10) Pub. No.: US 2026/0035424 A1
(43) Pub. Date: Feb. 5, 2026

(54) CYTOKINE-RECEPTOR FUSIONS FOR IMMUNE CELL STIMULATION

(71) Applicant: Caribou Biosciences, Inc., Berkeley, CA (US)

(72) Inventor: Rodolfo Gonzalez, Burlingame, CA (US)

(73) Assignee: Caribou Biosciences, Inc., Berkeley, CA (US)

(21) Appl. No.: 18/868,054

(22) PCT Filed: May 24, 2023
(86) PCT No.: PCT/US2023/067427

§ 371 Date: Nov. 21, 2024

Related U.S. Application Data
(60) Provisional application No. 63/346,045, filed on May 26, 2022.

(51) Int. Cl. C07K 14/54; C07K 14/715; C12N 5/0783; C12N 15/86

(52) U.S. Cl. CPC C07K 14/54; C07K 14/7155; C12N 5/0646; C12N 15/86; C07K 2319/02; C12N 2510/00; C12N 2750/14143

(57) ABSTRACT

A fusion protein of IL-21 cytokine and IL-21 receptor is disclosed. Also disclosed are cells comprising the cytokine-receptor fusion, and methods and compositions for the treatment of tumors utilizing the cytokine-receptor fusion and utilizing the cells expressing the cytokine-receptor fusion.

### CROSS-REFERENCE TO RELATED APPLICATIONS

This application claims priority to U.S. provisional application Ser. No. 63/346,045 filed on May 26, 2022.

### FIELD OF THE INVENTION

The invention related to the field of molecular biology, immunology, and cellular immunotherapy. More specifically, the invention relates to the use of engineered cytokine proteins

### STATEMENT REGARDING FEDERALLY SPONSORED RESEARCH

None.

### SEQUENCE LISTING

The instant application contains a Sequence Listing which has been submitted electronically in XML format and is hereby incorporated by reference in its entirety. Said XML copy, created on May 17, 2023, is named CB1045_30_SL.xml and is 16,383 bytes in size.

### BACKGROUND OF THE INVENTION

Natural killer (NK) cells are the cells of the immune system possessing innate anti-tumor activity. Attempts have been made to develop therapies utilizing NKs as anti-tumor effectors. For example, NK cells may be engineered to express a chimeric antigen receptor (CAR) against tumor targets. The major obstacles with NK-based anti-tumor therapy are limited persistence, poor in vivo proliferation in the recipient patient, and limited anti-tumor activity.

Cytokine stimulation has been shown to ameliorate these problems by enhancing survival and immune competency of NK cells. It has been proposed that prior to administering to a patient, NK cells be expanded in the presence of antigen-presenting cells (APC) engineered to express membrane-bound cytokines such as IL-15 and IL-21 or in the presence of soluble cytokines such as IL-2, IL-7, IL-12, IL-15, IL-12, and IL-21 (See U.S. Application Publication No. 20200390816). In another approach, cytokines are administered to the patient receiving a dose of anti-tumor NK cells. Unfortunately, cytokine administration is not without problems. First, a separate preparation of exogenous recombinant cytokines must be supplied. Second, as signaling molecules, soluble cytokines have a short in vivo half-life and require repeated administration. And lastly, third, soluble cytokines have inhibitory and even cytotoxic effects on other immune cells in the recipient patient. There is a need for an improved cytokine-based method to stimulate NK cells in the course of NK-based therapies.

### SUMMARY OF THE INVENTION

The invention comprises a fusion protein of IL-21 cytokine and IL-21 receptor, cells comprising the cytokine-receptor fusion and methods and composition for the treatment of tumors with the cytokine-receptor fusion and with the cells expressing the cytokine-receptor fusion protein.

In one embodiment, the invention is an isolated fusion protein comprising: a cytokine IL-21 domain; an amino acid linker; and an IL-21 receptor domain. In some embodiments, the cytokine IL-21 domain comprises SEQ ID NO: 1. In some embodiments, the cytokine IL-21 domain is encoded by a sequence comprising SEQ ID NO: 2. In some embodiments, the IL-21 receptor domain comprises SEQ ID NO: 3. In some embodiments, the IL-21 receptor domain is encoded by a sequence comprising SEQ ID NO: 4. In some embodiments, the amino acid linker comprises from about 5 to about 40 amino acid residues. In some embodiments, the amino acid linker comprises a sequence derived from an immunoglobulin selected from the group consisting of IgG, IgA, I IgD, IgE, and IgM. In some embodiments, the amino acid linker comprises a sequence derived from the CH1, CH2, CH3 domain of an immunoglobulin heavy chain. In some embodiments, the amino acid linker consists of SEQ ID NO: 5. In some embodiments, the amino acid linker is encoded by a sequence comprising SEQ ID NO: 6. In some embodiments, the isolated fusion protein further comprises a signal peptide. In some embodiments, the signal peptide is selected from a C2 signal peptide, and an IL-2 signal peptide. In some embodiments, the signal peptide comprises SEQ ID NO: 7. In some embodiments, the signal peptide is encoded by a sequence comprising SEQ ID NO: 8. In some embodiments, the isolated fusion protein comprises SEQ ID NO: 9. In some embodiments, the isolated fusion protein is encoded by a sequence comprising SEQ ID NO: 10.

In one embodiment, the invention is an isolated nucleic acid comprising a vector and a nucleotide sequence encoding the fusion protein comprising: a cytokine IL-21 domain; an amino acid linker; and an IL-21 receptor domain. In some embodiments, the isolated nucleic acid further comprises a promoter selected from the group consisting of PGK1 promoter, MND promoter, Ubc promoter, CAG promoter, CaMKIIa promoter, SV40 early promoter, SV40 late promoter, the cytomegalovirus (CMV) immediate early promoter, Rous sarcoma virus long terminal repeat (RSV-LTR) promoter, mouse mammary tumor virus long terminal repeat (MMTV-LTR) promoter, 0-interferon promoter, the hsp70 promoter EF-1a promoter, and R-Actin promoter. In some embodiments, the vector is a plasmid. In some embodiments, the vector is a viral vector derived from a virus selected from the group consisting of an adenovirus type 2 and an adenovirus type 5, a retrovirus, a lentivirus, an adeno-associated virus (AAV), a simian virus 40 (SV-40), vaccinia virus, Sendai virus, Epstein-Barr virus (EBV), and herpes simplex virus (HSV). In some embodiments, the isolated nucleic acid comprises SEQ ID NO: 10. In some embodiments, the vector is AAV6.

In one embodiment, the invention is an immune cell comprising the fusion protein comprising: a cytokine IL-21 domain; an amino acid linker; and an IL-21 receptor domain. In some embodiments, the immune cell is selected from a T-cell and a natural killer (NK) cell, and precursors thereof. In some embodiments, the NK cell is selected from a primary NK cell (pNK cell) and an induced NK cell (iNK cell). In some embodiments, the T cell is selected from the group consisting of a T-helper cell, a cytotoxic T cell and a regulatory T cell. In some embodiments, the immune cell comprises SEQ ID NO: 9. In some embodiments, the immune cell comprises SEQ ID NO: 10. In some embodiments, the immune cell further comprises a chimeric antigen receptor (CAR). In some embodiments, the CAR comprises an antigen binding region targeting a tumor antigen selected from the group consisting of CD19, CD371, CD269 (BCMA), CA-125, MUC-1, CD56, EGFR, c-Met, AKT, Her2, Her3, CD99, CLL-1, CD47, CD33, CS1, ROR1, c-Met, TROP2, EphA2, GD2, GPC3, epithelial tumor antigen, melanoma-associated antigen, mutated TP53, mutated Ras, and mutated BRAF. In some embodiments, the CAR comprises an intracellular domain selected from the group consisting of TCR zeta chain, CD3 zeta chain, CD28, CD27, OX40/CD134, 4-1BB/CD137, ICOS/CD278, IL-2Rbeta/CD122, IL-2Ralpha/CD132, DAP10, DAP12, DNAM1, TLR1, TLR2, TLR4, TLR5, TLR6, MyD88, CD40, and a combination thereof. In some embodiments, immune cell further comprises an armoring modification. In some embodiments, the armoring modification comprises inactivation of an immune checkpoint gene is selected from the group consisting of PDCD1 gene, CTLA-4, LAG3, Tim3, BTLA, BY55, TIGIT, B7H5, LAIR1, SIGLEC10, and 2B4. In some embodiments, the armoring modification comprises inactivation of the beta-2 microglobulin (B2M) gene. In some embodiments, the immune cell further comprises an immune cloaking modification. In some embodiments, the immune cloaking modification comprises an HLA-E-B2M fusion.

In one embodiment, the invention is a method of making an immune cell comprising introducing into the cell SEQ ID NO:10. In some embodiments, the introducing is via electroporation. In some embodiments, the introducing is via electroporation of naked DNA. In some embodiments, the introducing is via a vector. In some embodiments, the vector is a viral vector derived from a virus selected from the group consisting of an adenovirus type 2 and an adenovirus type 5, a retrovirus, a lentivirus, an adeno-associated virus (AAV), a simian virus 40 (SV-40), vaccinia virus, Sendai virus, Epstein-Barr virus (EBV), and herpes simplex virus (HSV). In some embodiments, the vector is AAV6. In some embodiments, the method further comprises introducing into the cell a sequence-dependent endonuclease. In some embodiments, the sequence-dependent endonuclease is introduced as part of a CRISPR system comprising a nucleic acid-guided endonuclease and nucleic acid-targeting nucleic acid (NATNA) guides. In some embodiments, the nucleic acid-guided endonuclease is selected from Cas9, Cas12a and CASCADE. In some embodiments, one or more components of the CRISPR system are introduced into the cell in the form of DNA. In some embodiments, one or more components of the CRISPR system are introduced into the cell in the form of RNA. In some embodiments, the CRISPR system is introduced into the cell in the form of a nucleoprotein complex. In some embodiments, the endonuclease comprises a catalytically inactive CRISPR endonuclease conjugated to the cleavage domain of the restriction endonuclease Fok I. In some embodiments, the endonuclease is selected from the group consisting of a zinc finger nuclease (ZFN), a ZFN-Fok I fusion, a transcription activator-like effector nuclease (TALEN), and a TALEN-Fok I fusion. In some embodiments, the endonuclease cleaves the genome of the cell at a locus selected from the group consisting of TRAC, CBLB, PDCD1, CTLA-4, LAG3, Tim3, BTLA, BY55, TIGIT, B7H5, LAIR1, SIGLEC10, and 2B4. In some embodiments, SEQ ID NO: 10 is inserted into a locus selected from the group consisting of TRAC, CBLB, PDCD1, CTLA-4, LAG3, Tim3, BTLA, BY55, TIGIT, B7H5, LAIR1, SIGLEC10, and 2B4.

In one embodiment, the invention is a composition comprising an immune cell comprising the fusion protein comprising: a cytokine IL-21 domain; an amino acid linker; and an IL-21 receptor domain, and a pharmaceutically acceptable excipient. In some embodiments, the pharmaceutically acceptable excipient comprises one or more of carbohydrates, inorganic salts, antimicrobial agents, antioxidants, surfactants, buffers, acids, bases, water, alcohols, polyols, glycerin, vegetable oils, phospholipids, surfactants, sugars, derivatized sugars, alditols, mannitol, xylitol, maltitol, lactitol, xylitol, sorbitol, pyranosyl sorbitol, myoinositol, aldonic acid, esterified sugars, sugar polymers, monosaccharides, fructose, maltose, galactose, glucose, D-mannose, sorbose, disaccharides, lactose, sucrose, trehalose, cellobiose, polysaccharides, raffinose, melezitose, maltodextrins, dextrans, starches, citric acid, sodium chloride, potassium chloride, sodium sulfate, potassium nitrate, and sodium phosphate. In some embodiments, the antimicrobial agent comprises one or more of benzalkonium chloride, benzethonium chloride, benzyl alcohol, cetylpyridinium chloride, chlorobutanol, phenol, phenylethyl alcohol, phenylmercuric nitrate, and thimerosal. In some embodiments, the composition further comprises an antioxidant selected from ascorbyl palmitate, butylated hydroxyanisole, butylated hydroxytoluene, hypophosphorous acid, monothioglycerol, propyl gallate, sodium bisulfite, sodium formaldehyde sulfoxylate, and sodium metabisulfite. In some embodiments, the composition further comprises a surfactant selected from polysorbates, sorbitan esters, lecithin, phosphatidylcholines, phosphatidylethanolamines, fatty acids, fatty acid esters and cholesterol. In some embodiments, the composition further comprises a freezing agent selected from 3% to 12% dimethylsulfoxide (DMSO) and 1% to 5% human albumin. In some embodiments, the composition further comprises a preservative selected from one or more of methylparaben, propylparaben, sodium benzoate, benzalkonium chloride, antioxidants, chelating agents, parabens, chlorobutanol, phenol, and sorbic acid.

In one embodiment, the invention is a method of inhibiting the growth of a tumor in a patient comprising administering to the patient the composition comprising an immune cell comprising the fusion protein comprising: a cytokine IL-21 domain; an amino acid linker; and an IL-21 receptor domain, and a pharmaceutically acceptable excipient. In some embodiments the tumor is selected from a solid tumor and a hematological tumor. In some embodiments the administering is selected from the group consisting of systemic delivery, including parenteral delivery, intramuscular, intravenous, subcutaneous, and intradermal delivery. In some embodiments the composition further comprises a delivery-timing component that enable time-release, delayed release, or sustained release of the composition. In some embodiments, the delivery-timing component is selected from monostearate, gelatin, a semipermeable matrix, and a solid hydrophobic polymer. In some embodiments, the method further comprises administering a cytokine to the patient. In some embodiments, the cytokine is selected from IL-2 and IL-15.

### BRIEF DESCRIPTION OF THE DRAWINGS

FIG. 1 is a diagram of one embodiment of an expression contract for expressing a protein comprising an IL-21/IL-21 receptor fusion.

FIG. 2 illustrates a proposed mechanism of action of the IL-21/IL-21 receptor fusion in a cell.

FIG. 3 is a diagram of a study design for accessing the effect of cytokines IL-2, IL-15 and IL-21 on treatment of tumor xenograft-injected mice with NK cells.

FIG. 4 is an experimental protocol used to access the effect of cytokines IL-2, IL-15 and IL-21 on treatment of tumor xenograft-injected mice with NK cells.

FIG. 5 shows the phenotype of the NK cells used in the study.

FIG. 6 shows an in vitro cytotoxicity assay of NK cells against SKOV3-Luc-eGFP tumor cells.

FIG. 7 shows percentage of NK cells in the live cell gated population recovered from the whole blood and peritoneal flush of xenograft-injected mice 7 days after administering the NK cells and different cytokine combinations.

FIG. 8 shows total recovery of NK cells from the xenograft-injected mice 7 days after administering the NK cells and different cytokine combinations.

FIG. 9 shows weekly measurements of tumor burden in xenograft-injected mice following administration of NK cells and different cytokine combinations (averaged within each treatment group).

FIG. 10 shows weekly measurements of tumor burden in xenograft-injected mice following administration of NK cells and different cytokine combinations (broken down by treatment groups).

FIG. 11 shows representative images of tumors in xenograft-injected mice following administration of NK cells and different cytokine combinations assessed by live animal fluorescence at day 53 post-engraftment.

FIG. 12 shows changes in body weight for each treatment group.

FIG. 13 shows survival of animals in each treatment group.

### DETAILED DESCRIPTION OF THE INVENTION

### Definitions

Unless defined otherwise, technical, and scientific terms used herein have the same meaning as commonly understood by a person of ordinary skill in the art. See, Sambrook et al., Molecular Cloning, A Laboratory Manual, 4^{th} Ed. Cold Spring Harbor Lab Press (2012).

The following definitions are provided to aid in understanding of the disclosure.

The term “therapeutic benefit” refers to an effect that improves the condition of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the tumor, or prevention of metastasis, or prolonging overall survival (OS) or progression free survival (PFS) of a subject with cancer.

The terms “pharmaceutically acceptable” and “pharmacologically acceptable” refer to molecular entities and compositions that do not produce an adverse, allergic, or other deleterious reaction in a patient. For example, the pharmaceutically and pharmacologically acceptable preparations should meet the standards set forth by the FDA Office of Biological Standards.

The term “pharmaceutically acceptable carrier” and “excipient” refer to aqueous solvents (e.g., water, aqueous solutions of alcohols, saline solutions, sodium chloride, Ringer's solution, etc.), non-aqueous solvents (e.g., propylene glycol, polyethylene glycol, vegetable oil, and injectable organic esters), as well as dispersion media, coatings, surfactants, gels, antioxidants, preservatives (e.g., antibacterial or antifungal agents, anti-oxidants, chelating agents, and inert gases), isotonic agents, absorption delaying agents, stabilizers, binders, disintegration agents, lubricants, sweetening agents, flavoring agents, and dyes. The concentration and pH of the various components in a pharmaceutical composition are adjusted according to well-known parameters for each component.

The term “domain” refers to one region in a polypeptide which is folded into a particular structure independently of other regions.

The term “effector function” refers to a specialized function of a differentiated cell, such as a NK cell.

The term “adoptive cell” refers to a cell that can be genetically modified for use in a cell therapy treatment. Examples of adoptive cells include lymphocytes, macrophages, and natural killer (NK) cells.

The term “cell therapy” refers to the treatment of a disease or disorder that utilizes genetically modified cells. The term “adoptive cell therapy (ACT)” refers to a therapy that uses genetically modified adoptive cells. Examples of ACT include T-cell therapies, CAR-T cell therapies, natural killer (NK) cell therapies and CAR-NK cell therapies.

The term “lymphocyte” refers to a leukocyte that is part of the vertebrate immune system. Lymphocytes include T-cells such as CD4+ and/or CD8+ cytotoxic T cells, alpha/beta T cells, gamma/delta T cells, and regulatory T cells. Lymphocytes also include natural killer (NK) cells, natural killer T (NKT) cells, cytokine induced killer (CIK) cells, and antigen presenting cells (APCs), such as dendritic cells. Lymphocytes also include tumor infiltrating lymphocytes (TILs).

The terms “effective amount” and “therapeutically effective amount” of a composition such as a cell therapy composition, refer to a sufficient amount of the composition to provide the desired response in the patient to whom the composition is administered.

The terms “peptide,” “polypeptide,” and “protein” are interchangeable and refer to polymers of amino acids, including natural and synthetic (unnatural) amino acids, as well as amino acids not found in naturally occurring proteins, e.g., peptidomimetics, and D optical isomers. A polypeptide may be branched or linear and be interrupted by non-amino acid residues. The terms also encompass amino acid polymers that have been modified through acetylation, disulfide bond formation, glycosylation, lipidation, phosphorylation, cross-linking, or conjugation (e.g., with a label). The polypeptide need not include the full-length amino acid sequence of the reference molecule but can include only so much of the reference molecule as necessary in order for the polypeptide to retain its desired activity. For example, polypeptides comprising full-length proteins, fragments thereof, polypeptides with amino acid deletions, additions, and substitutions are encompassed by the terms “protein” and “polypeptide,” as long as the desired activity is retained. For example, polypeptides with 95%, 90%, 80%, 70% or less of sequence identity with the reference polypeptide are included as long the desired activity is retained by the polypeptides.

The terms “CRISPR” (clustered regularly interspaced short palindromic repeats), “CRISPR-Cas” (CRISPR-associated protein) and “CRISPR system” refer to the genome editing tool derived from prokaryotic organisms and comprising a nucleic acid guide molecule and a sequence-specific nucleic acid-guided endonuclease capable of cleaving a target nucleic acid strand at a site complementary to a sequence in the nucleic acid guide.

The term “NATNA” (nucleic acid targeting nucleic acid) refers to a nucleic acid guide molecule of the CRISPR system. NATNA may be comprised two nucleic acid targeting polynucleotides (“dual guide”) including a CRISPR RNA (crRNA) and transactivating CRISPR RNA (tracrRNA). NATNA may be comprised a single nucleic acid targeting polynucleotide (“single guide”) comprising crRNA and tracrRNA connected by a fusion region (linker). The crRNA may comprise a targeting region and an activating region. The tracrRNA may comprise a region capable of hybridizing to the activating region of the crRNA. The term “targeting region” refers to a region that is capable of hybridizing to a sequence in a target nucleic acid. The term “activating region” refers to a region that interacts with a polypeptide, e.g., a CRISPR nuclease.

The current state of the art use of IL-21 stimulation in cancer immunotherapy involves administration of recombinant human IL-21 in both pre-clinical and clinical settings. More recent studies utilize a targeted antibody-based approach to deliver IL-21 to specific effector cells. For example, IL-21 has been fused to a monoclonal antibody targeting PD-1 (Li, Y., et al., (2021) Targeting IL-21 to tumor-reactive T cells enhances memory T cell responses and anti PD-1 antibody therapy, Nature Comm. 12:951). In a similar approach, IL-21 was fused to the monoclonal antibody Erbitux® for delivery to EGFR+ tumor cells and for presentation to cytotoxic T cells. (Deng, S., et al., (2020) Targeting tumors with IL-21 reshapes the tumor microenvironment by proliferating PD-1^{int} Tim3-CD8+ T cells, JCI Insight 5(7)e132000).

IL-21 (interleukin-21) is a type 1 cytokine secreted as a 133-amino acid protein by activated CD4+ T cells. IL-21 has been demonstrated to possess potent stimulatory effects on the proliferation, differentiation and activation of B-cells, T-cells, and NK-cells. The biological effects of IL-21 are mediated via the IL-21 receptor complex, which is composed of an IL-21 private receptor chain (IL-21 Rα) which is 538 amino acids long, in complex with the common gamma chain (ye), which is also a part of the other interleukin receptors including receptors for IL-2, IL-4, IL-7, IL-9, and IL-15. When bound to IL-21, the IL-21 receptor acts through the Jak/STAT pathway, utilizing Jak1, Jak3 and a STAT3 homodimer to activate its target genes.

IL-21 exerts its signaling through a heterodimeric receptor complex consisting of the IL-21 receptor (IL-21R) and the common γ-chain. IL-21R belongs to the family of class I cytokine receptors characterized by a signature motif (WSXWS) involved in C-mannosylation. The extracellular domain of IL-21R comprises two fibronectin domains connected by a sugar bridge. This domain anchors at the mannosylated WSXWS motif through hydrogen bonding. See Hamming, O., et al., (2012) Crystal structure of Interleukin 21 receptor (IL-21R) bound to IL-21 reveals that sugar chain interacting with WSXWS motif is integral part of IL-21R, J. Biol. Chem., 287(12) 9454.

Fusing a cytokine and its specific receptor has been accomplished with IL-15. Rowley, J. et al., ((2009) Expression of IL-15RA or an IL-15 IL-15RA fusion on CD8 T cells modifies adoptively transferred T cell function in cis. Eur. J. Immunol. 39:491) have successfully fused IL-15 with IL-15R via a serine-glycine linker and demonstrated improved viability and proliferation of CD8+ T cells expressing the fusion.

Disclosed herein is an invention comprising a fusion of IL-21 to its receptor IL-21R and the use of the fusion to stimulate immune cells including T cells and NK cells. Particular embodiments comprise methods and compositions for stimulating NK-cells, iNK cells and CAR-NK cells are disclosed. Also disclosed herein are methods of treating tumors by administering NK-cells, iNK cells or CAR-NK cells stimulated by or expressing the cytokine-receptor fusions of the invention.

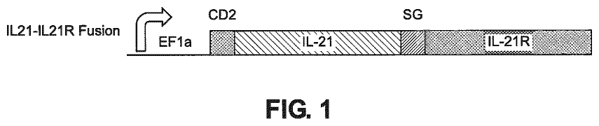

In some embodiments, the invention comprises a fusion protein having an amino acid sequence of IL-21 linked to the amino acid sequence of the IL-21 receptor. FIG. 1 is a diagram of an exemplary nucleic acid construct encoding the IL-21/IL-21R fusion. In some embodiments, the nucleic acid construct comprises an EF1a promoter, a sequence coding for the CD2 leader sequence, a sequence coding for TL-21, a sequence coding for the serine-glycine linker (SG in FIG. 1), and a sequence coding for the IL-21 receptor.

In some embodiments, IL-21 is a 133 amino acid protein represented by SEQ ID NO: 1. In some embodiments, IL-21 is encoded by a nucleic acid sequence of by SEQ ID NO: 2. In some embodiments, IL-21R is a 221 amino acid protein represented by SEQ ID NO: 3. In some embodiments, IL-21R is encoded by a nucleic acid sequence of by SEQ ID NO. 4. In some embodiments, the fusion protein comprises a peptide linker of SEQ ID NO: 5 encoded by SEQ ID NO: 6. In some embodiments, the fusion protein comprises a signal peptide. In some embodiments, the signal peptide selected from C2 and IL-2 signal peptide. In some embodiments, the signal peptide is the C2 signal peptide represented by SEQ ID NO: 7 and is encoded by SEQ ID NO: 8.

In some embodiments, the IL-21/IL-21 receptor fusion protein comprises SEQ ID NO: 9 encoded by the nucleic acid sequence of SEQ ID NO: 10.

In some embodiments, the fusion protein is an isolated fusion protein. In some embodiments, the fusion protein comprises in an N- to C-terminal orientation: IL-21 cytokine, an amino acid linker, and IL-21 receptor, where the linker comprises from about 5 to about 40 amino acids. The linker can be a naturally occurring or an engineered sequence. For example, in some embodiments, a linker is derived from a human protein, e.g., an immunoglobulin selected from IgG, IgA, I IgD, IgE, or IgM. In some embodiments, the linker comprises 5-40 amino acids from the CH1, CH2, CH3 domain of an immunoglobulin heavy chain. In some embodiments, the linker is a glycine- and serine-rich linkers exemplified by SGGGSGGGGSGGGGSGGGGSGGGSLQ (SEQ ID NO: 5) and additional sequences disclosed in the U.S. Pat. No. 5,525,491 Serine-rich peptide linkers, U.S. Pat. No. 5,482,858 Polypeptide linkers for production of biosynthetic proteins, and a publication WO2014087010 Improved polypeptides directed against IgE.

In some embodiments, the IL-21 portion of the fusion protein consists of SEQ ID NO: 1. In some embodiments, IL-21R portion of the fusion protein consists of SEQ ID NO: 3. In some embodiments, the serine-glycine linker consists of SEQ ID NO: 6. In some embodiments, the C2 signal peptide portion of the fusion protein consists of SEQ ID NO: 9. In some embodiments, the IL-21/IL-21R fusion protein consists of SEQ ID NO: 8.

In some embodiments, IL-21 portion of the nucleic acid construct consists of SEQ ID NO: 2. In some embodiments, IL-21R portion of the nucleic acid construct consists of SEQ ID NO. 4. In some embodiments, the serine-glycine linker portion of the nucleic acid construct consists of SEQ ID NO:5. In some embodiments, the C2 signal peptide portion of the nucleic acid construct consists of SEQ ID NO: 10. In some embodiments, the portion of the nucleic acid construct encoding the IL21/IL21R fusion protein consists of SEQ ID NO: 7.

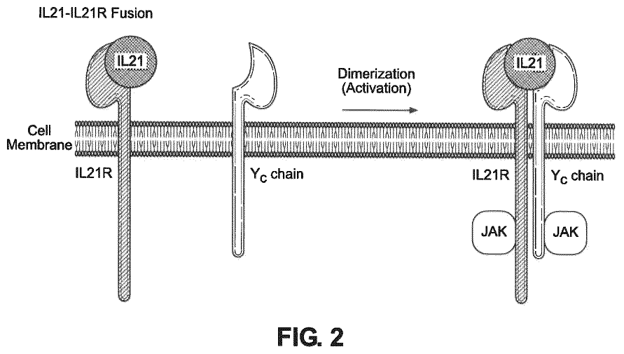

The function of the fusion protein is illustrated in FIG. 2. The IL-21/IL21-R fusion protein is translocated to the cell membrane whereby IL-21R assembles with the common cytokine receptor γ chain, γ_{c} to form the functional receptor complex. The IL-21 cytokine is permanently bound to the IL-21R-γ_{c} complex, and the fusion protein can interact with the downstream signaling pathways inside the cell. Without being bound by a particular theory, the inventors propose that the fusion protein activates the IL-21/IL-21R signaling cascade through JAK1 and JAK3.

In some embodiments, the invention comprises adoptive cells and the use of adoptive cells in cellular immunotherapy. Adoptive cells of the instant invention include lymphocytes, such as T cells, CAR-T cells, NK cells, iNK cells, CAR-NK cells, and tumor infiltrating lymphocytes (TILs). Other cells that can be used in the context of the invention are macrophages and stem cells including induced pluripotency stem cells (iPSCs), cord blood stem cells, and hematopoietic stem cells. The cells can be isolated from a healthy donor of from a human patient using standard techniques. For example, lymphocytes can be isolated from blood, solid tumors (in the case of TILs), or from lymphoid organs such as the thymus, bone marrow, lymph nodes, and mucosal-associated lymphoid tissues (MALT). Techniques for isolating lymphocytes from such tissues are well known in the art, see, e.g., Smith, J. W. (1997) Apheresis techniques and cellular immunomodulation, Ther. Apher. 1:203-206. In some embodiments, isolated lymphocytes are characterized in terms of specificity, frequency, and function. In some embodiments, the isolated lymphocyte population is enriched for specific subsets of T cells, such as CD4+, CD8+, CD25+, or CD62L+. See, e.g., Wang et al., Mol. Iherapy—Oncolytics (2016) 3:16015. In some embodiments, after isolation, lymphocytes are activated in order to promote proliferation and differentiation into specialized lymphocytes. For example, T cells can be activated using soluble CD3/28 activators, or magnetic beads coated with anti-CD3/anti-CD28 monoclonal antibodies.

In certain embodiments, NK cells are derived from a source selected from human peripheral blood mononuclear cells (PBMC), leukapheresis products (PBSC), bone marrow, or umbilical cord blood by methods well known in the art. Alternatively, NK cells can be obtained by differentiating human embryonic stem cells (hESCs) or induced pluripotency stem cells (iPSCs). NKs differentiated from iPSCs are referred to as iNK cells. The NK cells may be heterologous, autologous, or allogeneic. The heterologous NK cells may be haplotype matched for the subject in the HLA or KIR loci to be administered as cell therapy. Human NK cells can be identified by certain cell-surface markers, such as CD8, CD16, and CD56.

Methods of isolating NK cells from cord blood are known in the art, e.g., by Ficoll density gradient centrifugation, see Spanholtz, J. et al., (2011) Clinical-grade generation of active NK cells from cord blood hematopoietic progenitor cells for immunotherapy using a closed-system culture process, PloS one, 6(6), e20740, and Shah, N., et al., (2013) Antigen presenting cell-mediated expansion of human umbilical cord blood yields log-scale expansion of natural killer cells with anti-myeloma activity. PloS one, 8(10), e76781. In some embodiments, the isolated cell composition is depleted of CD3+ cells. In some embodiments, the isolated cell composition is enriched for CD56+ cells. In some embodiments, the isolated cell composition is enriched for CD45+ cells. In some embodiments, the isolated cell composition is enriched for CD56+/CD45+ cells. In some embodiments, a quality control measure or characterization step is applied to the isolated cell composition, e.g., determining the percentage of CD56⁺/CD3⁻, CD45⁺/CD3-cells, CD56⁺/CD45⁺, or CD56⁺/CD45⁺/CD3⁻ in the composition.

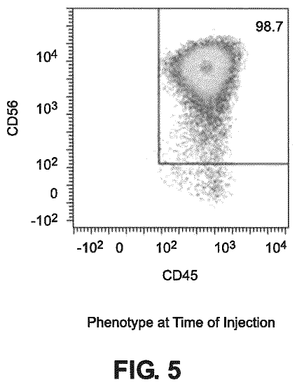

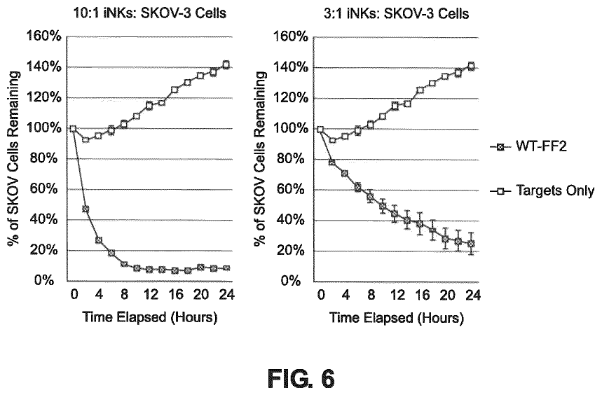

In some embodiments, the isolated cell composition is enriched for CD56⁺/CD45⁺ cells. In some embodiments, the quality control measure or characterization step is determining the percentage of CD56⁺/CD45⁻ cells in the composition by flow cytometry as illustrated in FIG. 5. In some embodiments, the quality control measure or characterization step is determining the ability to kill tumor cells in coculture. In some embodiments, the ability to kill tumor cells is determined in a cytotoxicity assay involving a coculture with the ratio of NK cells to tumor cells at between 10:1 and 3:1 and the decrease in tumor cell number is measured over a 24-hour period as illustrated in FIG. 6.

In some embodiments, the cells described herein are genetically modified to express a chimeric antigen receptor (CAR). In some embodiments, the cells are CAR-T cells. In some embodiments, the cells are CAR-NK cells derived from primary NK cells or from iNK cells. The CAR comprises an extracellular domain comprising an antigen binding region, a transmembrane domain and one or more intracellular co-activation (co-stimulatory) and activation (stimulatory) domains.

In some embodiments, the extracellular domain of the CAR comprising the antigen binding region targets a tumor antigen. In some embodiments, the tumor antigen is selected from CD-19, CD-371, CD-269 (BCMA), CA-125, MUC-1, CD56, EGFR, c-Met, AKT, Her2, Her3, CD99, CLL-1, CD47, CD33, CS1, ROR1, c-Met, TROP2, EphA2, GD2, GPC3, epithelial tumor antigen, melanoma-associated antigen, or a mutated protein selected from TP53, Ras and BRAF. In some embodiments, the antigen binding region of the CAR is derived from a monoclonal antibody. In some embodiments, the antigen binding region comprises a fragment of the variable portion of the heavy chain or the light chain (V_{H} or V_{L}) of a single-chain variable fragment (scFv) derived from a particular monoclonal antibody. The single-chain variable fragment (scFv) has the ability to bind to an antigen. The scFV is comprised of the Fv regions of immunoglobulin heavy chain (H chain) and light chain (L chain) linked via a spacer sequence.

In some embodiments, the transmembrane domain of the CAR is derived from a membrane-bound or transmembrane protein. For example, the transmembrane domain of the CAR may be the transmembrane domain of a T cell receptor alpha-chain or beta-chain, a CD3-zeta chain, CD28, CD3-epsilon chain, CD45, CD4, CD5, CD8, CD9, CD16, CD22, CD33, CD37, CD64, CD80, CD86, CD134, CD137, ICOS, CD154, DNAM1, NKp44, NKp46, NKG2D, 2B4, or a GITR.

The intracellular signaling domain of a CAR is responsible for activation of one or more effector functions of the immune cell expressing the CAR. In some embodiments, the intracellular signaling domain of the CAR comprises a part of or the entire sequence of the TCR zeta chain, CD3 zeta chain, CD28, CD27, OX40/CD134, 4-1BB/CD137, ICOS/CD278, IL-2Rbeta/CD122, IL-2Ralpha/CD132, DAP10, DAP12, DNAM1, TLR1, TLR2, TLR4, TLR5, TLR6, MyD88, CD40 or a combination thereof.

In some embodiments, the CAR is a fully human or is humanized to reduce immunogenicity in human patients. In some embodiments, the CAR sequence is optimized for codon usage in human cells.

The nucleic acid encoding the CAR may be introduced into a cell as a genomic DNA sequence or a cDNA sequence. The cDNA sequence comprises the open reading frame for the translation of the CAR and in some embodiments, further comprises untranslated elements that improve for example, the stability or the rate of translation of the CAR mRNA.

In some embodiments, the cells used in the invention further comprise a genome modification resulting in armoring of the cells against an attack by the immune system of a recipient of the allogeneic immune cells. In some embodiments, the armoring modification comprises protection from recognition by the cytotoxic T cells of the host. Cytotoxic T cells recognize MHC Class I antigen. MHC Class I molecule is comprised of beta-2 microglobulin (B2M) associated with heavy chains of HLA-I proteins (selected from HLA-A, HLA-B, HLA-C, HLA-E, HLA-F and HLA-G) on the surface of the cell. The B2M/HLA-I complex on the surface of the allogeneic cell is recognized by cytotoxic CD8+ T cells and if HLA-I is recognized as non-self, allogeneic cell is killed by the T cells. In some embodiments, the cells of the invention comprise an armoring genomic modification comprising a disruption of the B2M gene and therefore, disruption of the MHC Class I antigen recognition and cytotoxic T cell attack.

In some embodiments, the armoring genome modification comprises disruption of recognition by the NK cells of the host. NK cells recognize cells without MHC-I protein as “missing self” and kill such cells. NK cells are inhibited by HLA-I molecules, including HLA-E, a minimally polymorphic HLA-I protein. In some embodiments, the cells of the invention comprise a first armoring genomic modification comprising a disruption of the B2M gene and therefore, disruption of the MHC Class I antigen recognition and cytotoxic T cell attack, and further comprise a second armoring genomic modification comprising an insertion of an HLA-E gene fused to beta-2-microglobulin (B2M) gene, and therefore, expression of the HLA-E/B2M construct and cloaking the cells from an attack by NK cells. See, e.g., Gornalusse et al., (2017) HLA-E-expressing pluripotent stem cells escape allogeneic responses and lysis by NK cells, Nat. Biotechnol. (2017) 35:765-772.

In some embodiments, the armoring modification comprises transcriptionally silencing or disrupting one or more immune checkpoint gene. In some embodiments, the one or more immune checkpoint gene is selected from PD1 (encoded by the PDCD1 gene), CTLA-4, LAG3, Tim3, BTLA, BY55, TIGIT, B7H5, LAIR1, SIGLEC10, and 2B4 as disclosed in the U.S. application publication US20150017136 Methods for engineering allogeneic and highly active T cell for immunotherapy.

In some embodiments, the invention comprises a method of producing the fusion protein of the invention. In some embodiments, the fusion protein is produced in vitro and isolated to yield an isolated fusion protein. In some embodiments, the nucleic acid encoding the protein is introduced into a target cell where expression of the fusion protein is desired. In some embodiments, the introduced nucleic acid is selected from an expression vector and an RNA encoding the fusion protein. In some embodiments, the target cells are contacted with the nucleic acid encoding the protein in vitro, in vivo or ex vivo.

Methods for producing engineered proteins are known in the art. For example, DNA molecules encoding the cytokine-receptor fusion described herein can be formed in vitro through recombinant DNA methods from isolated human sequences or can be chemically synthesized using the sequence information provided herein. Synthetic DNA molecules can be inserted into vectors comprising all the necessary sequences for transcription and translation of the inserted DNA sequence in the desired host cell.

In some embodiments, the vector is a viral vector (e.g., a retroviral vector, adenoviral vector, adeno-associated viral vector, or lentiviral vector). Suitable vectors are non-replicating in the target cells. In some embodiments, the vector is selected from or designed based on SV40, EBV, HSV, or BPV. The vector incorporates the protein expression sequences. In some embodiments, the expression sequences are codon-optimized for expression in mammalian cells. In some embodiments, the vector also incorporates regulatory sequences including transcriptional activator binding sequences, transcriptional repressor binding sequences, enhancers, introns, and the like. In some embodiments, the viral vector supplies a constitutive or an inducible promoter. In some embodiments, the promoter is selected from EF1α, PGK1, MND, Ubc, CAG, CaMKIIa, and β-Actin promoter. In some embodiments, the promoter is selected from the SV40 early and late promoters, the cytomegalovirus (CMV) immediate early promoter, and the Rous sarcoma virus long terminal repeat (RSV-LTR) promoter, mouse mammary tumor virus long terminal repeat (MMTV-LTR) promoter, the β-interferon promoter, the hsp70 promoter and EF-1a promoter. In some embodiments, the promoter is an EF-1a promoter.

In some embodiments, the viral vector supplies a transcription terminator.

In some embodiments, the vector is a plasmid selected from a prokaryotic plasmid, a eukaryotic plasmid, and a shuttle plasmid.

In some embodiments, the fusion protein described herein is expressed in a prokaryotic cell and the vector is a plasmid comprising a prokaryotic promoter, and a prokaryotic signal sequence. In some embodiments, the fusion protein described herein is expressed in a eukaryotic cell and the vector is a plasmid comprising a eukaryotic promoter active in the desired cell type, a secretion signal, a poly-A sequence, and a stop codon, and, optionally, one or more regulatory elements such as enhancer elements.

In some embodiments, the expression vector comprises one or more selection marker. In some embodiments, the selection markers are antibiotic resistance genes or other negative selection markers. In some embodiments, the selection markers comprise proteins whose mRNA is transcribed together with the fusion protein mRNA and the polycistronic transcript is cleaved prior to translation.

In some embodiments, the expression vector comprises polyadenylation sites. In some embodiments, the polyadenylation sites are SV-40 polyadenylation sites.

In some embodiments, the coding sequence of the cytokine-receptor fusion is introduced into the cells via a viral vector, such as e.g., AAV vector (AAV6) or any other suitable viral vector capable of delivering an adequate payload. In some embodiments, to facilitate homologous recombination, the coding sequence is joined to homology arms located 5′ (upstream) and 3′ (downstream) of the insertion site in the desired insertion site in the genome. In some embodiments, the homology arms are about 500 bp long. In some embodiments, the sequence coding for the cytokine-receptor fusion together with the homology arms are cloned into a viral vector plasmid. The plasmid is used to package the sequences into a virus.

In some embodiment, the cells such as T-cells or NK cells or precursors thereof are contacted with a viral vector so that the genetic material delivered by the vector is integrated into the genome of the target cell and then expressed in the cell or on the cell surface. Transduced and transfected cells can be tested for transgene expression using methods well known in the art such as fluorescence-activated cell sorting (FACS), microfluidics-based screening, ELISA, or Western blot. For example, IL-21/IL-21 receptor fusion expressing CAR-NK cells can be tested by staining of flow cytometry with IL-21 or IL-21R specific antibodies or a combination of the IL-21 and IL-21R specific antibodies.

The present invention involves manipulating nucleic acids, including genomic DNA and plasmid DNA that were isolated or extracted from a sample. Methods of nucleic acid extraction are well known in the art. See J. Sambrook et al., “Molecular Cloning: A Laboratory Manual,” 1989, 2nd Ed., Cold Spring Harbor Laboratory Press: New York, N.Y.). A variety of reagent and kits are commercially available for extracting nucleic acids (DNA or RNA) from biological samples, including products from BD Biosciences (San Jose, Cal.), Clontech (TaKaRa Bio.); Epicentre Technologies (Madison, Wisc.); Gentra Systems, (Minneapolis, Minn.); Qiagen (Valencia, Cal.); Ambion (Austin, Tex.); BioRad Laboratories (Hercules, Cal.); KAPA Biosystems (Roche Sequencing Solutions, Pleasanton, Cal.) and more.

In some embodiments, the invention involves intermediate purification or separation steps for nucleic acids, e.g., to remove unused reactants from the DNA. The purification or separation may be performed by a size selection method selected from gel electrophoresis, affinity chromatography and size exclusion chromatography. In some embodiments, size selection can be performed using Solid Phase Reversible Immobilization (SPRI) technology from Beckman Coulter (Brea, Cal.).

In some embodiments, exogenous protein-coding nucleic acid sequences (e.g., IL-21/IL-21 receptor fusion-coding sequences of the instant invention, CAR-coding sequences, etc.) are introduced into a cell such as an NK cell or T cell or precursors thereof. In some embodiments, the “naked” nucleic acids are introduced into lymphocytes by electroporation as described e.g., in U.S. Pat. No. 6,410,319.

In some embodiments, the cell comprises the CRISPR system. In some embodiments, the CRISPR system comprises a nucleic acid-guided endonuclease and nucleic acid-targeting nucleic acid (NATNA) guides (e.g., a CRISPR guide RNAs selected from tracrRNA, crRNA or a single guide RNA incorporating the elements of the tracrRNA and crRNA in a single molecule). In some embodiments, the components of the CRISPR system are introduced into the cells (e.g., NK cells or T cells or precursors thereof) in the form of nucleic acids.

In some embodiments, the components of the CRISPR system are introduced into the cells (e.g., NK cells or T cells or precursors thereof) in the form of DNA coding for the nucleic acid-guided endonuclease and NATNA guides. In some embodiments, the gene coding for the nucleic acid-guided endonuclease (e.g., a CRISPR nuclease selected from Cas9 and Cas12a) is inserted into a plasmid capable of propagating in the target cell. In some embodiments, the gene coding for the NATNA guides is inserted into a plasmid capable of propagating in the target cell.

In some embodiments, the nucleic acid-guided endonuclease and NATNA guides are introduced into the target cells (e.g., NK cells or T cells or precursors thereof) in the form of RNA, e.g., the mRNA coding for the nucleic acid-guided endonuclease along with the NATNA guides.

In some embodiments, the nucleic acid-guided endonuclease and the NATNA guides are introduced into the target cells (e.g., NK cells or T cells or precursors thereof) as a preassembled nucleoprotein complex. In some embodiments, the nucleic acid-guided endonuclease and the NATNA guides are introduced into the target cells (e.g., NK cells or T cells or precursors thereof) via any combination of different means, e.g., the endonuclease is introduced as the DNA via a plasmid containing the gene encoding the endonuclease while the guides are introduced in its final format as RNA (or RNA containing DNA nucleotides).

In some embodiments, the nucleic acids encoding the nucleic acid-guided endonuclease and NATNA guides are introduced into the target cells via electroporation.

In some embodiments, the nucleic acids coding for the nucleic acid-guided endonuclease are introduced into cells in the form of mRNA as described e.g., in the U.S. Pat. No. 10,584,352 via electroporation of viral pseudo-transduction as described therein.

In some embodiments, one or more of the coding sequences described herein are introduced into the genome of the cell using a sequence-specific endonuclease. In some embodiments, the endonuclease is a nucleic acid-guided endonuclease encoded by the CRISPR locus. The CRISPR (Clustered Regularly Interspaced Short Palindromic Repeats) genomic locus is found many prokaryotic genomes and provides resistance to invasion of foreign nucleic acids. Structure, nomenclature and classification of CRISPR loci are reviewed in Makarova et al., Evolution and classification of the CRISPR-Cas systems. Nature Reviews Microbiology. 2011 June; 9(6): 467-477.

Briefly, a typical CRISPR locus includes a number of short repeats regularly interspaced with spacers. The CRISPR locus also includes coding sequences for CRISPR-associated (Cas) genes. A spacer-repeat sequence unit encodes a CRISPR RNA (crRNA). In vivo, a mature crRNAs are processed from a polycistronic transcript referred to as pre-crRNA or pre-crRNA array. The repeats in the pre-crRNA array are recognized by Cas-encoded proteins that bind to and cleave the repeats liberating mature crRNAs. CRISPR systems perform cleavage of a target nucleic acid wherein Cas proteins and crRNA form a CRISPR ribonucleoproteins (crRNP). The crRNA molecule guides the crRNP to the target nucleic acid (e.g., a foreign nucleic acid invading a bacterial cell) and the Cas nuclease proteins cleave the target nucleic acid.

Type I CRISPR systems include means for processing the pre-crRNA array that include a multi-protein complex called CASCADE (CRISPR-associated complex for antiviral defense) comprised of subunits CasA, B, C, D and E. The Cascade-crRNA complex recognizes the target nucleic acid through hybridization of the target nucleic acid with crRNA. The bound nucleoprotein complex recruits the Cas3 helicase/nuclease to facilitate cleavage of target nucleic acid.

Type II CRISPR systems include a trans-activating CRISPR RNA (tracrRNA). The tracrRNA hybridizes to a crRNA repeat in the pre-crRNA array and recruits endogenous RNaseIII to cleave the pre-crRNA array. The tracrRNA/crRNA complex can associate with a nuclease, e.g., Cas9. The crRNA-tracrRNA-Cas9 complex recognizes the target nucleic acid through hybridization of the target nucleic acid with crRNA. Hybridization of the crRNA to the target nucleic acid activates the Cas9 nuclease. for target nucleic acid cleavage.

Type III CRISPR systems include the RAMP superfamily of endoribonucleases (e.g., Cas6) that cleave the pre-crRNA array with the help of one or more CRISPR polymerase-like proteins.

Type V CRISPR systems comprise a different set of Cas-like genes, including Csf1, Csf2, Csf3 and Csf4 which are distant homologues of Cas genes in Type I-III CRISPR systems.

CRISPR endonucleases require a nucleic acid targeting nucleic acid (NATNA) also known as guide RNAs. The endonuclease is capable of forming a ribonucleoprotein complex (RNP) with one or more guide RNAs. In some embodiments, the endonuclease is a Type II CRISPR endonuclease and NATNA comprises tracrRNA and crRNA.

In some embodiments, NATNA is selected from the embodiments described in U.S. Pat. No. 9,260,752. Briefly, a NATNA can comprise, in the order of 5′ to 3′, a spacer extension, a spacer, a minimum CRISPR repeat, a single guide connector, a minimum tracrRNA, a 3′ tracrRNA sequence, and a tracrRNA extension. In some instances, a nucleic acid-targeting nucleic acid can comprise, a tracrRNA extension, a 3′ tracrRNA sequence, a minimum tracrRNA, a single guide connector, a minimum CRISPR repeat, a spacer, and a spacer extension in any order.

In some embodiments, the guide nucleic acid-targeting nucleic acid can comprise a single guide NATNA. The NATNA comprises a spacer sequence which can be engineered to hybridize to the target nucleic acid sequence. The NATNA further comprises a CRISPR repeat comprising a sequence that can hybridize to a tracrRNA sequence. Optionally, NATNA can have a spacer extension and a tracrRNA extension. These elements can include elements that can contribute to stability of NATNA. The CRISPR repeat and the tracrRNA sequence can interact, to form a base-paired, double-stranded structure. The structure can facilitate binding of the endonuclease to the NATNA.

In some embodiments, the single guide NATNA comprises a spacer sequence located 5′ of a first duplex which comprises a region of hybridization between a minimum CRISPR repeat and minimum tracrRNA sequence. The first duplex can be interrupted by a bulge. The bulge facilitates recruitment of the endonuclease to the NATNA. The bulge can be followed by a first stem comprising a linker connecting the minimum CRISPR repeat and the minimum tracrRNA sequence. The last paired nucleotide at the 3′ end of the first duplex can be connected to a second linker connecting the first duplex to a mid-tracrRNA. The mid-tracrRNA can comprise one or more additional hairpins.

In some embodiments, the NATNA can comprise a double guide nucleic acid structure. The double guide NATNA comprises a spacer extension, a spacer, a minimum CRISPR repeat, a minimum tracrRNA sequence, a 3′ tracrRNA sequence, and a tracrRNA extension. The double guide NATNA does not include the single guide connector. Instead, the minimum CRISPR repeat sequence comprises a 3′ CRISPR repeat sequence and the minimum tracrRNA sequence comprises a 5′ tracrRNA sequence and the double guide NATNAs can hybridize via the minimum CRISPR repeat and the minimum tracrRNA sequence.

In some embodiments, NATNA is an engineered guide RNA comprising one or more DNA residues (CRISPR hybrid RDNA or chRDNA). In some embodiments, NATNA is selected from the embodiments described in U.S. Pat. No. 9,650,617. Briefly, some chRDNA for use with a Type II CRISPR system may be composed of two strands forming a secondary structure that includes an activating region composed of an upper duplex region, a lower duplex region, a bulge, a targeting region, a nexus, and one or more hairpins. A nucleotide sequence immediately downstream of a targeting region may comprise various proportions of DNA and RNA. Other chRDNA may be a single guide D(R)NA for use with a Type II CRISPR system comprising a targeting region, and an activating region composed of and a lower duplex region, an upper duplex region, a fusion region, a bulge, a nexus, and one or more hairpins. A nucleotide sequence immediately downstream of a targeting region may comprise various proportions of DNA and RNA. For example, the targeting region may comprise DNA or a mixture of DNA and RNA, and an activating region may comprise RNA or a mixture of DNA and RNA.

In some embodiments, the endonuclease used to introduce one or more of the genetic modifications described herein (e.g., gene inactivation or insertion of the cytokine-receptor fusion-coding sequences of the instant invention, CAR-coding sequences, armoring sequences such as B2M-HLA-I protein fusions) into the genome of a cell is a restriction endonuclease, e.g., a Type II restriction endonuclease.

In some embodiments, the endonuclease used to introduce one or more of the genetic modifications described herein is a catalytically inactive CRISPR endonuclease (e.g., catalytically inactive Cas9 or Cas12a) conjugated to the cleavage domain of the restriction endonuclease Fok I. (see e.g., Guilinger, J. P., et al., (2014). Fusion of catalytically inactive Cas9 to FokI nuclease improves the specificity of genome modification, Nature biotechnology, 32(6), 577-582.

In some embodiments the endonuclease the endonuclease used to introduce one or more of the genetic modifications described herein is a zinc finger nuclease (ZFN), or a ZFN-Fok I fusion. In such embodiments, the target sequence is about 22-52 bases long and comprises a pair of ZFN recognition sequences, each 9-18 nucleotides long, separated by a spacer, which is 4-18 nucleotides long. (see e.g., Kim Y. G., et al., (1996). Hybrid restriction enzymes: zinc finger fusions to FokI cleavage domain, Proc Natl Acad Sci USA. 93(3): 1156-1160.

In some embodiments, the endonuclease the endonuclease used to introduce one or more of the genetic modifications described herein is a transcription activator-like effector nuclease (TALEN), or a TALEN-Fok I fusion. In such embodiments, the target sequence is about 48-85 nucleotides long and comprises a pair of TALEN recognition sequences, each 18-30 bases long, separated by a spacer, which is 12-25 bases long. (see e.g., Christian M. et al., (2010) Targeting DNA double-strand breaks with TAL effector nucleases, Genetics. 186 (2): 757-61.

In some embodiments, the engineered IL-21/IL-21R fusion protein disclosed herein is inserted into a double-strand break in the genome of the cell. In some embodiments, the introduction of the engineered protein coincides with inactivation of another gene by the insertion of the engineered fusion protein (gene knock-out and simultaneous gene knock-in). In some embodiments, the insertion site and an inactivated gene is TRAC, CBLB, PDCD1, CTLA-4, LAG3, Tim3, BTLA, BY55, TIGIT, B7H5, LAIR1, SIGLEC10, and 2B4.

In some embodiments, the invention comprises compositions including cells such as NK cells or CAR-NK cells or precursors thereof, engineered to express the cytokine-receptor fusion of IL-21/IL21R described herein. Once produced, the engineered cells can be formulated into compositions for delivery to the subject to be treated. The compositions include the engineered lymphocytes, and one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerin, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

In some embodiments, the composition further comprises an antimicrobial agent for preventing or deterring microbial growth. In some embodiments, the antimicrobial agent is selected from benzalkonium chloride, benzethonium chloride, benzyl alcohol, cetylpyridinium chloride, chlorobutanol, phenol, phenylethyl alcohol, phenylmercuric nitrate, thimerosal, and combinations thereof.

In some embodiments, the composition further comprises an antioxidant added to prevent the deterioration of the lymphocytes. In some embodiments, the antioxidant is selected from ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

In some embodiments, the composition further comprises a surfactant. In some embodiments, the surfactant is selected from polysorbates, sorbitan esters, lipids, such as phospholipids (lecithin and other phosphatidylcholines), phosphatidylethanolamines, fatty acids and fatty esters; steroids, such as cholesterol.

In some embodiments, the composition further comprises a freezing agent such as 3% to 12% dimethylsulfoxide (DMSO) or 1% to 5% human albumin.

The number of adoptive cells, such as NK cells or CAR-NK cells, in the composition will vary depending on a number of factors but will optimally be a therapeutically effective dose per vial. A therapeutically effective dose can be determined experimentally by repeated administration of increasing amounts of the composition in order to determine which amount produces a clinically desired endpoint.

In some embodiments, the invention is a method of treating, preventing, or ameliorating a disease, or condition comprising administering a population of cells (T-cells or NK cells, or CAR-T cells or CAR-NK cells or precursors thereof) expressing the cytokine-receptor fusion of IL-21/IL21R described herein.

In diseases or conditions that can be treated by the cytokine fusion expressing cells of the disclosure include various malignancies. In some embodiments, the malignancies or tumors are solid tumors selected from melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, esophageal cancer, gastric cancer, gastrointestinal stromal tumors (GIST), colorectal cancer, pancreatic cancer, prostate cancer, testicular cancer, bladder cancer, ovarian cancer, liver cancer, hepatocellular carcinoma, cholangiocarcinoma, brain and central nervous system cancer, neuroendocrine cancer, thyroid cancer, parathyroid cancer, and endometrial cancer, uterine cancer, sarcoma, and kidney cancer. In some embodiments, the malignancies or tumors are hematological tumors selected from Hodgkin's lymphoma, non-Hodgkin's lymphoma, leukemia, and multiple myeloma.

In some embodiments, the invention is a method of inhibiting the growth of a tumor in a patient.

In some embodiments, the invention comprises a method of administering to a subject a therapeutically effective number of adoptive cells expressing the IL-21/IL21R cytokine-receptor fusion described herein. In some embodiments, the adoptive cells are NK cells or CAR-NK cells. In some embodiments, the adoptive cells are pre-activated and expanded prior to administration. In some embodiment, the administration of the adoptive cells according to the invention results in treating, preventing, or ameliorating the disease or condition in the subject. In some embodiments, the disease or disorder is selected from cancers or tumors and infection that can be treated by administration of immune cells that elicit an immune response.

A pharmaceutical composition comprising cells expressing the cytokine receptor fusion of the present disclosure can be delivered via various routes and delivery methods such as local or systemic delivery, including parenteral delivery, intramuscular, intravenous, subcutaneous, or intradermal delivery.

In some embodiments, for example, where the subject is a human, the dose includes fewer than about 1×10⁸ of CAR-expressing cells. In some embodiments, the cell therapy comprises administration of a dose comprising about 1×10⁵ cells/kg to 5×10⁶ cells/kg of body weight of the subject.

In some embodiments, the composition of the present invention is administered to a subject who has been preconditioned with an immunodepleting (e.g., lymphodepleting) therapy. In some embodiments, preconditioning is with lymphodepleting agents, including combinations of cyclosporine and fludarabine,

In some embodiments, the composition or formulation for administering to the patient is a pharmaceutical composition or formulation which permits the biological activity of an active ingredient and contains only non-toxic additional components such as pharmaceutically acceptable carriers. In some embodiments, pharmaceutically acceptable carriers include buffers, excipients, stabilizers, and preservatives.

In some embodiments, a preservative is used. In some embodiments, the preservative comprises one or more of methylparaben, propylparaben, sodium benzoate, benzalkonium chloride, antioxidants, chelating agents, parabens, chlorobutanol, phenol, and sorbic acid. In some embodiments, the preservative is present at about 0.0001% to about 2% by weight of the total composition.

In some embodiments, a carrier is used. In some embodiments, the carrier comprises a buffer, antioxidants including ascorbic acid and methionine; proteins, such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the carrier comprises a buffer. In some embodiments, the buffer comprises citric acid, sodium citrate, phosphoric acid, potassium phosphate, and various other acids and salts. In some embodiments, the buffer is present at about 0.001% to about 4% by weight of the total composition.

In some embodiments, the pharmaceutical composition delivery systems such that the delivery of the composition occurs over time. In such embodiments the pharmaceutical composition comprises release-timing components. In some embodiments, the pharmaceutical composition comprises aluminum monostearate or gelatin. In some embodiments, the pharmaceutical composition comprises semipermeable matrices of solid hydrophobic polymers. In some embodiments, the matrices are in the form of films or microcapsules.

In some embodiments, the pharmaceutical composition comprises a sterile liquid such as an isotonic aqueous solution, suspension, emulsion, dispersions, or viscous composition, which may be buffered to a selected pH. In some embodiments, the pharmaceutical composition is a sterile injectable solution prepared by incorporating the cells in a solvent such as sterile water, physiological saline, or solutions or glucose, dextrose, or the like. In some embodiments, the pharmaceutical composition further comprises dispersing, or emulsifying agents, pH buffering agents, gelling or viscosity enhancing additives, preservatives, flavoring agents, colors, and the like, depending upon the route of administration and the preparation desired.

In some embodiments, the adoptive cells (T-cells or NK cells, or CAR-T cells or CAR-NK cells or precursors thereof) expressing the IL-21/IL-21R cytokine-receptor fusion described herein are co-administered with additional cytokines. In some embodiments, the additional cytokines are selected from IL-2 or IL-15. In some embodiments, the cytokines are administered at 10 ng/mouse for IL-15 and 100,000 units/mouse for IL-2 or at an equivalent dose per kg of body weight of a human.

### EXAMPLES

### Example 1. Assessing iNK Cells Prior to Administration

iNK cells at the day of injection were stained with anti-human CD45-FITC and anti-human CD56-alexa647 and analyzed by flow cytometry. Results are shown in FIG. 5.

iNK cells were further assessed in a cytotoxicity assay. iNK cells were co-cultured with SKOV3-Luc-eGFP cells at either 10:1 (left) or 3:1 (right) ratio of iNK cells to SKOV3-Luc-eGFP cells and imaged every 2 hours using the Incucyte microscope (IntelliCyt/Essen BioScience, Inc.) Ann Arbor, Mich.). The data is displayed as the percent of live SKOV3-Luc-eGFP cells remaining relative to the initial number at the onset of the cytotoxicity assay. In a control assay, SKOV3-Luc-eGFP cells were cultured in the absence of iNK cells. Error bars are standard error of the mean of technical triplicate wells used for analysis.

### Example 2. Cytokine IL-21 in Combination with IL-2 and IL-15 Co-Administered with iNKs in a Murine Ovarian Cancer Xenograft Model

In this example, we evaluated the role of IL-21 and other cytokines and cytokine combinations on antitumor potency of iNK cells. The iNK cells are natural killer (NK) cells derived from inducible pluripotency stem cells (iPSC). We co-administered iNK cells with combinations of IL-2+IL-15, IL-2+IL-21, or IL-2+IL-15+IL-21 in an immunodeficient (NSG) mouse model. The mice were injected intraperitoneally with a luciferase-expressing SKOV3-Luc-eGFP ovarian cancer cell line and dosed with iNK cells.

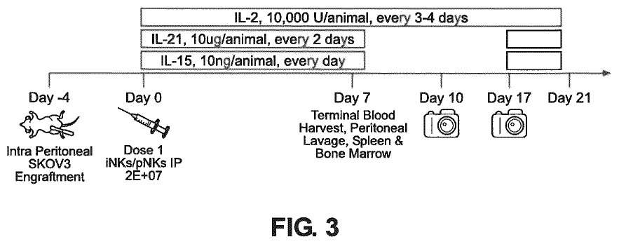

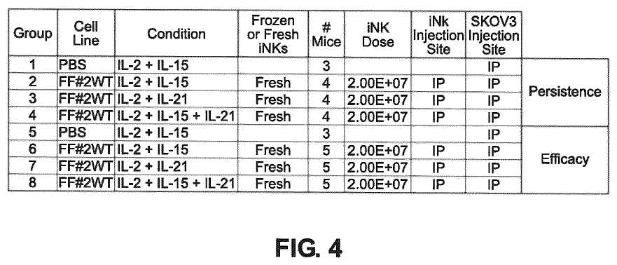

The experimental set up is shown FIG. 3 and FIG. 4. Briefly, NSG mice were injected intraperitoneally with 3×10⁵ SKOV3-Luc-eGFP tumor cells (ATCC number: HTB-77). After four days (at day zero) the mice were injected intraperitoneally with a dose of 2×10⁷ iNK cells. Simultaneously, cytokine administration was started in combinations shown in FIG. 4. For animals receiving IL-15, administration was at 10 ng/animal every day. For animals receiving IL-21, administration was at 10 μg/animal every 2 days. For animals receiving IL-2, administration was at 10,000 units/animal every 3-4 days. (FIG. 3).

The animals were split in two groups: the “persistence group” (1-4, FIG. 4) was sacrificed at day 7, and the “efficacy group” (5-8, FIG. 4) was monitored for at least 53 days and assessed weekly for tumor burden.

### Example 3. Measurement of iNK Cell Representation and Persistence in the Intraperitoneal Cavity and Whole Blood of SKOV3-Luc-eGFP Engrafted NSG Mice Upon iNK Cell and Cytokine Co-Administration

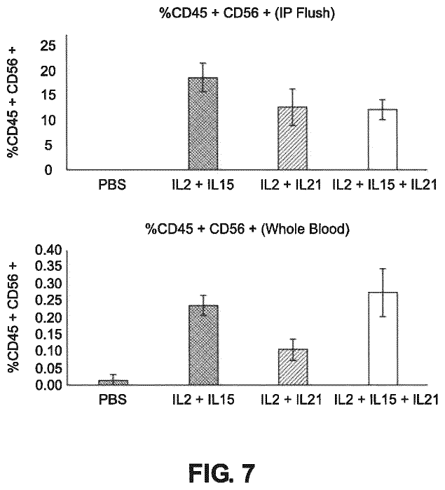

As a first assessment, at day 7, the animals of the “persistence group” were sacrificed. Intraperitoneal cavity flush and whole blood were collected from mice and processed using Ammonium-Chloride-Potassium lysis buffer to remove red blood cells, then stained using anti-human CD45-FITC and anti-human CD56-alexa647 and analyzed by flow cytometry. The data is presented as percent CD45+CD56+ within the live cell gated population based on 7-AAD negative staining. Results are shown in FIG. 7. Error bars are standard error of the mean. n=4 mice for each cytokine treatment group and n=3 for PBS group.

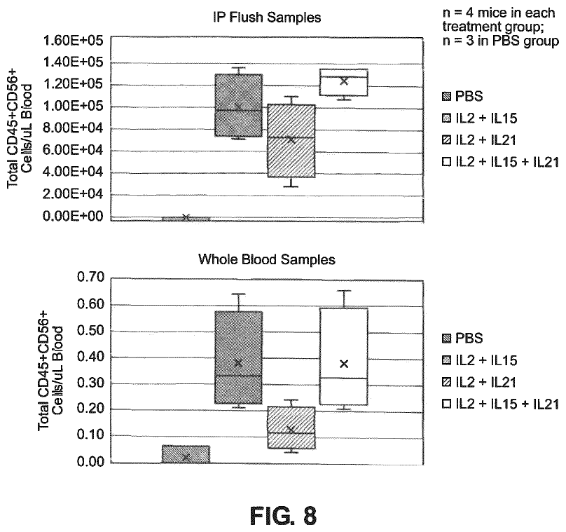

To quantify iNK cell persistence, flow cytometry counting beads were incorporated into the sample to allow for cell enumeration. Results are shown in FIG. 8. The data is presented as the total CD45+CD56+ cells (IP flush) or total CD45+CD56+ cells per μL of whole blood. The group sizes were n=4 mice for each cytokine treatment group, and n=3 for the PBS group.

### Example 4. Measurement of Tumor Burden of SKOV3-Luc-eGFP-Engrafted NSG Mice Upon iNK Cell and Cytokine Co-Administration

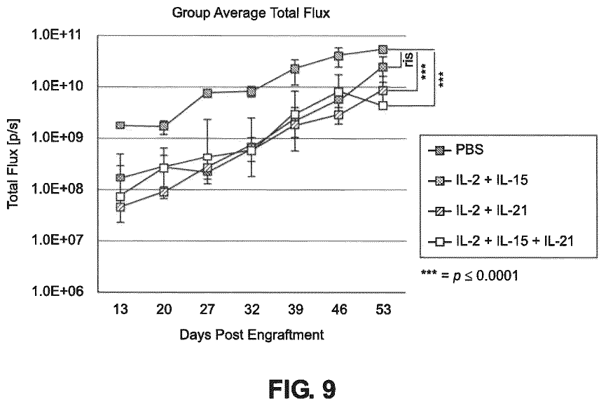

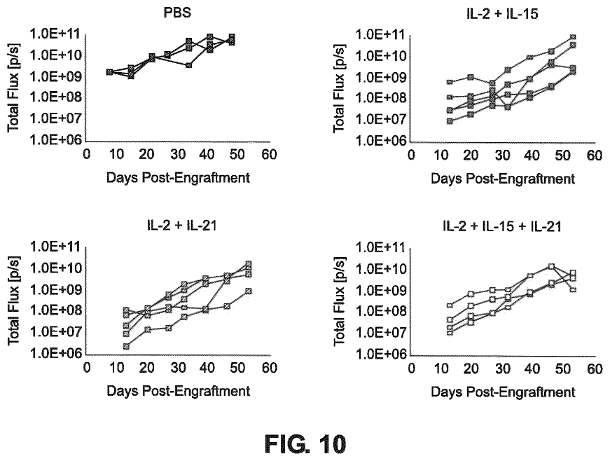

In another assessment, tumor burden of SKOV3-Luc-eGFP in mice with co-administration of iNK cells and the indicated combination of cytokines was measured weekly between days 13 and 53 post-engraftment. The data is presented as the mean total flux (photons per second) for each treatment group. Results are shown in FIG. 9 and FIG. 10. The data is presented as the mean total flux (photons per second) for each treatment group. The group sizes were n=5 mice for each cytokine treatment group and n=3 for PBS group. Significance values are calculated using one-way ANOVA. ***=p≤0.0001.

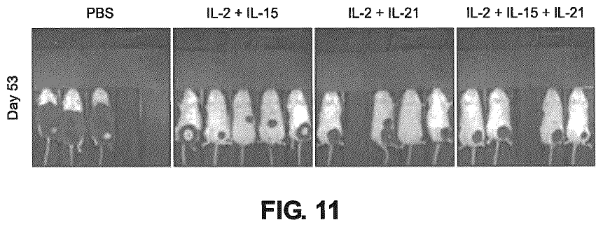

The animals were also imaged by IVIS@Spectrum in vivo imaging system that captures fluorescence of the SKOV3-Luc-eGFP tumors in live animals. (Perkin Elmer, Santa Clara, Cal.) Representative images of animals from each group are shown in FIG. 11.

### Example 5. Body Weight Measurement in SKOV3-Luc-eGFP Engrafted NSG Mice Upon iNK Cell and Cytokine Co-Administration

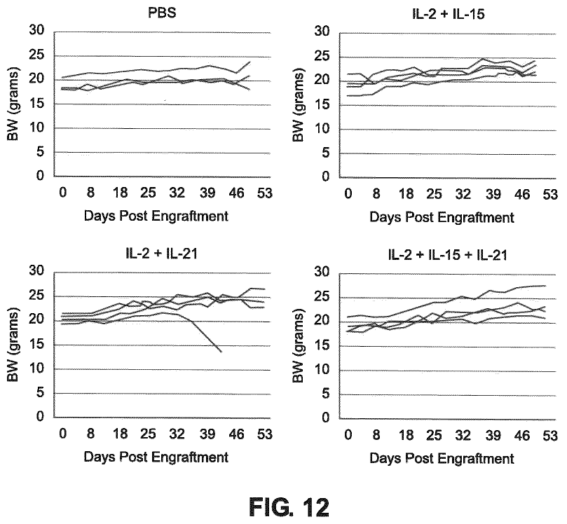

Body weight was measured weekly starting from day zero. Results are shown in FIG. 12. The group sizes were n=5 mice for each cytokine treatment group, and n=3 for PBS group at the onset of the study.

### Example 6. Probability of Survival of SKOV3-Luc-eGFP Engrafted NSG Mice Upon iNK Cell and Cytokine Co-Administration

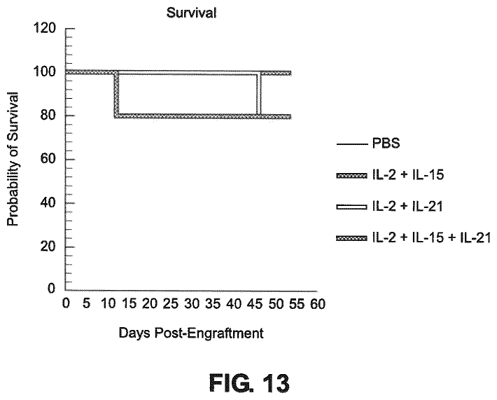

Survival of the NSG mice engrafted with SKOV3-Luc-eGFP tumor cells and receiving injections of iNK and a cytokine regimen was documented. Results are shown in FIG. 13 as Kaplan-Meier curves. The group sizes were n=5 mice for each cytokine treatment group, and n=3 for PBS group at the onset of the study.

### Example 7. Preparation of the IL-2/IL-21 Receptor Fusion Nucleic Acid Construct

The nucleic acid representing the IL-21/IL-21R fusion (SEQ ID NO: 10) was inserted in to a plasmid vector pCB7207 derived from pRCCB-CMV-Cas9-2A-Blast (Cellecta, Inc., Mountain View, Cal.) The portion containing the CMV promoter-Cas9-Blast resistance marker in the plasmid was replaced with the fragment containing EF1a promoter—IL-21/IL-21R-2A translation switch—Hygromycin resistance gene—WPRE terminator sequences.

### Example 8 (Prophetic). Nucleofection of iNK Cells with Cas12a-Guide Nucleoprotein Complexes

This example describes the nucleofection of iNK cell with a Cas12a-RNA guide nucleoprotein complex essentially as described in the International Application Publication No. WO2022086846.

The Cas12a sequence is cloned from Acidaminococcus spp. (strain BV3L6), conjugated to a sequence coding for a nuclear localization signal (NLS), codon-optimized for and expressed in E. coli.

The Cas12a RNA guides are produced by linking the activating region of the Cas12a crRNA to a 20-nt targeting region capable of binding to the target sequence in the human TRAC gene or the human CBLB gene. The targeting region is capable of binding to a target sequence that occurs downstream (in a 3′ direction) of a 5′-TTTV-3′ protospacer adjacent motif (PAM) recognized by the Acidaminococcus spp. Cas12a endonuclease. Optionally, the guide molecule is CRISPR hybrid (R)DNA guide (chRDNA), wherein the targeting region comprised one or more DNA nucleotides as described in WO2022086846.

The Cas12a protein and the RNA (or chRDNA) guides are combined in vitro to form a nucleoprotein complex. The nucleoprotein complex is transfected into iNK cells using the Nucleofector™ 96-well Shuttle System (Lonza, Allendale, N.J.). The Cas12a-guide nucleoprotein complex is dispensed in a 2.5 μl final volume into individual wells of a 96-well plate. The suspended iNK cells are pelleted by centrifugation for 10 minutes at 200×g, washed with calcium and magnesium-free phosphate buffered saline (PBS), and the cell pellet is resuspended in 10 ml of calcium and magnesium-free PBS. The cells were counted using the Countess® II Automated Cell Counter (Life Technologies, Grand Island, N.Y.). Aliquots of 2.2×10⁷ cells are transferred to a 15 ml conical tube and pelleted. The PBS is aspirated, and the cells are resuspended in Nucleofector™ P4 or P3 solution (Lonza) to a density of 2×10⁵-10⁶ cells/ml in each sample. 20 μl of the cell suspension is then added to each well containing 2.5 μl of the Cas12a-guide nucleoprotein complexes, and the entire volume from each well is transferred to a well of a 96-well Nucleocuvette™ Plate (Lonza). The plate is loaded onto the Nucleofector™ 96-well Shuttle and cells are nucleofected using the CA137 Nucleofector™ program (Lonza). Post-nucleofection, 77.5 μl of ImmunoCult-XF complete medium (STEMCELLS Technologies, Cambridge, Mass.) supplemented with IL-2 (100 units/mL) is added to each well, and the entire volume of transfected cell suspension is transferred to a 96-well cell culture plate containing 100 μl pre-warmed ImmunoCult-XF complete medium supplemented with IL-2 (100 units/mL), transferred to a tissue culture incubator and maintained at 37° C. in 5% C02 for 48 hours before downstream analysis.

### Example 9 (Prophetic.) Preparation and Delivery of the Donor Sequence Comprising the IL-21 IL-21R Fusion

This Example describes cloning of the IL-21/IL-21 receptor fusion (SEQ ID NO: 10) from the plasmid vector of Example 7 into an AAV vector, production of the fusion-containing AAV, and transduction of the iNK cells with the fusion-containing AAV for site-specific integration of the IL-21/IL-21R fusion into the cellular genome.

To facilitate homologous recombination, 500 bp-long homology arms located 5′ (upstream) and 3′ (downstream) of the insertion site in the genome are identified. The 5′ and 3′ homology arms are appended to SEQ ID NO: 10 in a reverse orientation (i.e., 3′ to 5′) relative to the homology arms. The resulting construct is cloned into a recombinant AAV (rAAV) plasmid. The plasmid is used to package the sequences into an AAV6 virus.

The fusion-containing AAV6 is transduced into the cells containing the Cas12a-guide nucleoprotein complex from Example 8. The cells are infected with AAV6 at an MOI of 10⁶ at 4 hours after nucleofection with the Cas12a nucleoprotein complex. The cells are cultured in ImmunoCult-XF complete medium supplemented with IL-2 (100 units/mL) for 24 hours after the transductions. The next day, the transduced cells are transferred to 50 mL conical tubes and centrifuged at 300×g for approximately 7-10 minutes to pellet the cells. The supernatant is discarded, and the pellet is gently resuspended, in ImmunoCult-XF complete medium with IL-2.

While the invention has been described in detail with reference to specific examples, it will be apparent to one skilled in the art that various modifications can be made within the scope of this invention. Thus, the scope of the invention should not be limited by the examples described herein, but by the claims presented below.

1. An isolated fusion protein comprising:
(i) a cytokine IL-21 domain;
(ii) an amino acid linker; and
(iii) an IL-21 receptor domain.

2. The isolated fusion protein of claim 1, wherein the cytokine IL-21 domain comprises SEQ ID NO: 1 and the IL-21 receptor domain comprises SEQ ID NO: 3.

3. The isolated fusion protein of claim 1, wherein the cytokine IL-21 domain is encoded by a sequence comprising SEQ ID NO: 2 and the IL-21 receptor domain is encoded by a sequence comprising SEQ ID NO: 4.

4. (canceled)

5. (canceled)

6. (canceled)

7. The isolated fusion protein of claim 1, wherein the amino acid linker comprises a sequence derived from an immunoglobulin selected from the group consisting of IgG, IgA, I IgD, IgE, and IgM.

8. The isolated fusion protein of claim 7, wherein the amino acid linker comprises a sequence derived from the CH1, CH2, CH3 domain of an immunoglobulin heavy chain.

9. The isolated fusion protein of claim 1, wherein the amino acid linker consists of SEQ ID NO: 5.

10-14. (canceled)

15. The isolated fusion protein of claim 1 comprising SEQ ID NO: 9 with a cleavable signal peptide comprises SEQ ID NO: 7.

16. The isolated fusion protein of claim 1 encoded by a sequence comprising SEQ ID NO: 10.

17. An isolated nucleic acid comprising a vector and a nucleotide sequence encoding the fusion protein of claim 1.

18-22. (canceled)

23. An immune cell comprising the fusion protein of claim 1.

24.-32. (canceled)

33. The immune cell of claim 23, wherein the armoring modification comprises inactivation of an immune checkpoint gene is selected from the group consisting of PDCD1 gene, CTLA-4, LAG3, Tim3, BTLA, BY55, TIGIT, B7H5, LAIR1, SIGLEC10, beta-2 microglobulin (B2M), and 2B4.

34-36. (canceled)

37. A method of making the immune cell of claim 23, the method comprising introducing into a cell SEQ ID NO:10.

38.-52. (canceled)

53. A composition comprising the cell of claim 23 and a pharmaceutically acceptable excipient.

54-66. (canceled)
